(19) United States
(12) Patent Application Publication
(10) Pub. No.: US 2011/0172502 A1
(43) Pub. Date: Jul. 14, 2011

(54) DIARY/PLANNER FOR BREAST SURGERY PREPARATION AND METHOD

(75) Inventor: Lenore A. Carleton, Ventura, CA (US)

(73) Assignee: ALLERGAN, INC., Irvine, CA (US)

(21) Appl. No.: 13/006,365

(22) Filed: Jan. 13, 2011

Related U.S. Application Data
(63) Continuation of application No. 11/521,788, filed on Sep. 15, 2006.
(60) Provisional application No. 60/817,135, filed on Jun. 28, 2006.

(51) Int. Cl. A61B 5/00

(52) U.S. Cl. 600/300

(57) ABSTRACT

A breast augmentation or reconstruction surgery diary/planner and method includes a binder including a information section, disposed in said binder, regarding what a patient needs to know regarding surgical risks a self assessment form, disposed in said binder, for completion by said patient in order to facilitate discussion between a surgeon and said patient; and a surgery consent section, disposed in said binder, with signature blocks for both said surgeon and said patient.

### RELATED APPLICATIONS

This is a continuation application of U.S. patent application Ser. No. 11/521,788, filed Sep. 15, 2006, which claims the benefit of U.S. provisional patent application Ser. No. 60/817,135, filed Jun. 28, 2006, the entirety of each of which is incorporated by reference herein.

### FIELD OF THE INVENTION

Breast augmentation, also called augmentation mammaplasty, involves surgical placement of an implant behind each breast to increase its volume and enhance shape. Often, after weight loss, childbirth, or a result of aging, the breasts loose volume and their shape changes. In addition, many women choose to have their breast enlarge in order to satisfy their desire for a fuller bust line. Breast augmentation is preferably performed after the age of 22. In addition, revision surgery may also be performed in order to correct or improve the result of an original primary breast augmentation surgery.

Breast reconstruction includes primary reconstruction to replace the breast tissue that has been removed due to cancer or trauma or that has failed to develop properly due to a severe breast abnormality. Breast reconstruction also includes revision surgery to correct or improve the result of an original primary breast reconstruction surgery.

The present invention is directed to a patient diary and planner which describes the risk of surgery and provides a means of assessing the patients understanding of the risks to facilitate discussion with a plastic surgeon as part of a preoperative consultation.

### SUMMARY OF THE INVENTION

A breast augmentation reconstruction surgery diary and planner in accordance with the present invention generally includes a binder along with an information section disposed within the binder, regarding what a patient needs to know regarding surgical risks.

The bound diary may also include an acceptance of risk section for the surgeon to review with the patient after which both initial. In addition, the binder includes a surgery consent section with signature box for both the surgeon and the patient.

More particularly, the diary and planner in accordance with the present invention may further include a record section disposed within the binder for recording surgery dates, surgery location, and contact persons. A tracking section may be provided in the binder which includes device implant tracking instructions along with a device of tracking a form. Still more particularly, the binder may include a warranty section which describes optional warranty programs.

A method in accordance with the present invention for preparing and planning for breast augmentation or reconstruction includes a distributing a patient diary to a patient with information regarding what the patient needs to know regarding surgical risks and a self-assessment form for completion by the patient.

Method further includes consulting the patient to review the self-assessment form and assess the patient's understanding of the risks and obtaining a patient's signature on a consent to surgery form.

More particularly, the method may include consulting with the patient one to two weeks after completion of the self-assessment form.

Further, distributing a patient diary may include distribution a diary including sections to record surgical dates, surgical location, and contact persons. In addition, the distribution may include a section describing device tracking and patient's rights/responsibility. Further, the distribution may include distributing a diary which includes a section regarding optional warranty programs.

### BRIEF DESCRIPTION OF THE DRAWINGS

The advantages and features of the present invention will be better understood by the following description when considered in conjunction with the accompanying drawings, in which:

FIG. 1 is a perspective view of a diary and planner in accordance with the present invention of generally showing a binder along with sections therein; and

FIG. 2 is an illustration of a device tracking form.

### DETAILED DESCRIPTION

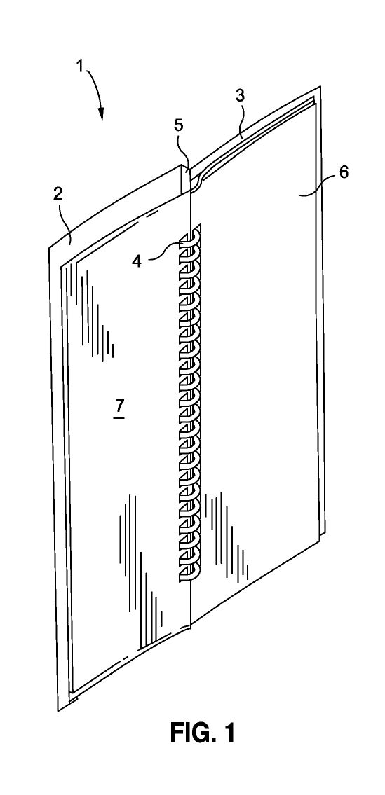

With reference to FIG. 1, there is shown a breast augmentation or reconstruction surgery diary and planner 1 generally including a binder 2 between a front cover 3 and a back cover 4 along with a spine 5 therebetween.

Multiple sections 6, 7 are illustrated. These sections 6, 7 among others will be described hereinafter.

The sections, not specifically referred to by character references, include an information section regarding what a patient needs to know regarding surgical risks. This section would include the following:

### Important Factors you Should Consider in Choosing Silicone-Filled Implants

Breast implants are not lifetime devices. Either because of rupture, other complications, or unacceptable cosmetic outcomes, you will likely need to have your implants removed at least once over your lifetime. When you have your implants replaced (revision-augmentation), your risk of future complications may increase compared to first time (primary) augmentation surgery. Because you will likely have revision-augmentation over the course of your lifetime, you should review the complication rates for revision-augmentation patients to see what future risk rates you may experience.
Whether you are undergoing primary augmentation or revision-augmentation, breast implantation is likely not a one-time surgery. You will likely need additional surgeries. These additional surgeries can include implant removal with or without replacement, or they can include other surgical procedures.
Breast implants may affect your ability to breast feed, such as reducing milk production.
Rupture of silicone-filled breast implant is most often silent. This means that neither your nor your surgeon will know that your implants have a rupture most of the time. In fact, the ability of physical examination by a plastic surgeon who is familiar with breast implants to detect silicone breast implant rupture is 30% compared to 89% for MRI.
After breast augmentation, you will need to have regular screening MRI examinations over your lifetime in order to determine if silent rupture is present. You should have your first MRI at 3 years after your initial implant surgery and then every 2 years thereafter. The cost of MRI screening may exceed the cost of your initial surgery over your lifetime. The cost should be considered in making your decision. These regular screenings do not replace any MRI recommendations your doctor may make relating to your individual care (for example, if you have a suspected rupture).
If implant rupture is noted on MRI, you should consider having the implant removed after consultation with your surgeon.
With breast implants, routine screening mammography will be more difficult. You should continue to perform monthly breast examinations for cancer screening; however, this may be more difficult. The implant may interfere with finding breast cancer during mammography. Because the breast and implant is squeezed during mammography, an implant may rupture during the procedure. More x-ray views are necessary for women with breast implants; therefore, you will receive more exposure to radiation. However, the benefit of having the mammogram to finding cancer outweighs the risk of the additional x-rays. Be sure to inform the mammography technologist that you have implants.
You should perform an examination of your implants every month. You should ask your surgeon to help you distinguish the implant from your breast tissue. The presence of lumps, persistent pain, swelling, hardening, or change in implant shape may be signs of symptomatic rupture of the implant. These should be reported to your surgeon and possibly evaluated with an MRI to screen for rupture.
Many of the changes to your breast following implantation are irreversible (cannot be undone). If you later choose to have your implant(s) removed and not replaced, you may experience dimpling, puckering, wrinkling, or other cosmetic changes of the breast.
After undergoing breast augmentation surgery (either primary or revision), your health insurance premiums may increase, your insurance coverage may be dropped, and/or future coverage may be denied. Treatment of complications may not be covered as well. You should discuss the complete extent of your insurance coverage with your insurance company before undergoing surgery.
You should inform any other doctor that treats you of the presence of implants to minimize the risk of damage to the implants.
Closed capsulotomy (use of pressure to force to “break up” the capsule) should not be used to treat capsular contracture. Closed capsulotomy can cause implant rupture.
Smoking may interfere with the healing process.
The long-term safety and effectiveness of INAMED implants has not been established. INAMED's Core Study is ongoing and is not concluded. The Core Study will continue to evaluate the long term (10 years) performance of these products. Unanswered questions remain regarding breast implants long-term. INAMED will continue to further address other long-term issues post-approval.
It is important that you read this entire diary/planner 1 because you need to understand the risks and benefits and to have realistic expectations of your outcome.

### Breast Implant Complications

Undergoing any type of surgical procedure involves risks (some serious) such as the effects of anesthesia, infection, swelling, redness, bleeding, pain, and even death, which need to be balanced against the benefits of the breast augmentation surgery. There are potential complications specific to breast implant surgery and breast implants, as described below. It should also be noted that the cited references include augmentation and/or reconstruction patients.

### What are the Primary Potential Complications

### Rupture

Breast implants are not lifetime devices. Breast implants rupture when the shell develops an opening. Rupture can occur at any time after implantation, but they are more likely to occur the longer the implant is implanted. Silicone-filled breast implant ruptures are most often silent. (MRI examination is currently the best method to screen for silent rupture.) This means that neither you nor your plastic surgeon will know if the implant has an opening in the shell most of the time. This is why MRI is recommended at 3 years and then every 2 years thereafter, to screen for rupture. Sometimes there are symptoms associated with gel implant rupture. These symptoms include hard knots or lumps surrounding the implant or in the armpit, flattening loss of size of the breast or implant, pain, tingling, swelling numbness, burning, or hardening of the breast.

The following things may cause your implant to rupture: damage by surgical instruments; stressing the implant during implantation and weakening it; folding or wrinkling of the implant shell; excessive force to the chest (for example, during closed capsulotomy, which is contraindicated); trauma; compression during mammogrpahic impaging; and severe capsular contracture. Breast implants may also simply wear out over time. Laboratory studies have identified some of the types of rupture; however, it is not known whether these tests have identified all causes of rupture. These laboratory studies will continue postapproval.

If rupture occurs, silicone gel may either remain within the scar tissue capsule surrounding the implant (intracapsular rupture) or move outside the capsule (extracapsular rupture). In some ruptures, gel may move beyond the breast. In studies of women evaluated with MRI involving a variety of manufactures and implant models, about three-fourths of implant ruptures are intracapsular and the remaining one-fourth are extracapsular.

There is a possibility that rupture may progress from intracapsular to extracapsular and beyond.

Studies of women indicate that over a 2-year period, about 10% of the implants with intra-capsular rupture progressed to extracapsular rupture as detected by MRI. This means that for women with silicone gel rupture within the scar tissue capsular detected via MRI after 2 years. 1 in 10 of these women will have progression of the gel outside the scar tissue capsule. In about half of these cases of progression from intra-to extra-capsular rupture, the women had had trauma or mammography. In the other half, no cause was given.

In the women with extracapsular rupture, after 2 years, the amount of silicone seepage outside the scar tissue capsule increased for about 14% of these women. This means that for 100 women with silicone gel rupture outside the scar tissue capsule, the amount of gel outside the scar tissue capsule increased for 14 women 2 years later.

There have been rare reports of gel movement to nearby tissues such as the chest wall, armpit, or upper abdominal wall, and to more distant locations down the arm or into the groin. This has led to damage to nerve bundles, granuloma formation and/or breakdown of tissues in direct contact with the gel in a few cases. There have been reports of silicone presence in the liver of patients with silicone breast implants. Movement of silicone gel material to lymph nodes in the axilla also has been reported, even in women without evidence of rupture, leading to lymphadenopathy, as discussed below. These reports were in women who had implants from a variety of manufactures and implant models.

Local breast complications reported in the published literature which were associated with rupture include breast hardness, a change in breast shape or size, and breast pain.

Concerns have been raised over whether ruptured implants are associated with the development of connective tissue or rheumatic diseases and/or symptoms such as fatigue and fibromylagia. A number of epidemiology studies have evaluated large populations of women with breast implants. These studies do not, taken together, support an association of breast implants and a diagnosed rheumatic disease. Other than one small study, these studies do not distinguish whether the women had ruptured or intact implants.

When MRI findings of rupture are found (such as subcapsular lines, characteristic folded wavy lines, teardrop sign, keyhole sign, noose sign), or if your surgeon believes you have signs or symptoms of rupture, you should have the implant and any gel removed. It also may be necessary to remove the tissue capsule as well as the implant, which will involve additional surgery, with associated costs. If you have symptoms such as breast hardness, a change in breast shape or size, and/or breast pain, you should have an MRI to determine whether rupture is present.

### Capsular Contracture

The scar tissue (capsule) that normally forms around the implant may tighten over time and compress the implant, making it feel firm and leading to what is called capsular contracture. Capsular contracture may be more common following infection, hematoma, and seroma, and the chance of it happening may increase over time. Capsular contracture occurs two times more commonly in revision-augmentation than in primary augmentation. Because you will likely have your initial implants replaced, you should be aware that your risk of capsular contracture may increase with revision-augmentation.

Capsular contracture may be a risk factor for implant rupture, and it is the most common reason for reopertation.

Symptoms of capsular contracture range from mild firmness and mild discomfort to severe pain, distorted shape of the implant, and palpability (ability to feel the implant).

Capsular contracture is graded into 4 levels depending on its severity. Grades III or IV are considered severe and often additional surgery is needed to correct these grades:
Grade I: the breast is normally soft and looks natural
Grade II: the breast is a little firm but looks normal
Grade III: the breast is firm and looks abnormal
Grade IV: the breast is hard, painful, and looks abnormal

In an INAMED Core Study, for women receiving augmentation implants for the first time, the risk of severe capsular contracture was 13% through 4 years. This means that 13 out of every 100 women who received INAMED's implants for primary breast augmentation had severe capsular contracture at least once during the first 4 years after receiving the implants.

For women receiving revision-augmentation implants, the risk of severe capsular contracture was 17% through 4 years. This means that 17 out of every 100 women who received INAMED implants for breast revision-augmentation had severe capsular contracture at least once during the first 4 years receiving the implants.

Additional surgery may be needed in cases where pain and/or firmness are severe. This surgery ranges from removal of the implant capsule tissue, to removal and possible replacement of the implant itself. This surgery may result is loss of your breast tissue. Capsular contracture may happen again after these additional surgeries. Capsular contracture may increase the risk of rupture.

In the study, capsular contracture was the most common reason for reoperation. Of the 103 women undergoing reoperations following primary breast augmentation in the study, 29% of the additional surgical procedures were to correct capsular contracture through 4 years. In addition, among the women in the study who had their implants removed through 4 years, 35% of the implant removals were because of capsular contracture.

For women with revision-augmentation implants in the study, capsular contracture was the most common reason for reoperation. Of the 39 women getting operations following revision breast augmentation in study, 17% of the additional surgical procedures were to correct capsular contracture through 4 years. In addition, among the women in the study who had their implants removed through 4 years, 28% of the implant removals were because of capsular contracture.

### Additional Surgies (Reoperations)

You should assume that you will need to have additional surgeries (reoperations). In one study, the operation rate was 24% for primary augmentation patients, which means that 24 out of every 100 women who received implants for primary augmentation had a reoperation during the first 4 years after receiving the implants. The reoperation rate was 35% for revision-augmentation patients, which means that 35 out of every 100 women who received implants for revision-augmentation had a reoperation during the first 4 years after receiving the implants.

Patients may decide to change the size or type of their implants, requiring additional surgery. Problems such as rupture, capsular contracture, hypertrophic scarring (irregular, raised scar), asymmetry, infection, and shifting can require additional surgery.

### Implant Removal

Because there are no lifetime devices, the longer you have your implants, the more likely it will be for you to have them removed for any reason, either because of dissatisfaction, an unacceptable cosmetic result, or a complication such as severe capsular contracture. Having your implants removed and replaced may increase your chances of getting future complications.

For women receiving primary augmentation implants in one study, 10% had their implants removed at lest once through 4 years. Severe capsular contracture and patient choice were the most common reasons for implant removal.

For women receiving revision-augmentation implants in the study, 13% had their implants removed at least once through 4 years. The most common reasons for implant removal were severe capsular contracture and patient choice.

Most women who have their implants removed, have them replaced with new implants, but some women do not. If you choose not to replace your implants, you may have cosmetically unacceptable dimpling, puckering, wrinkling, and/or other potentially permanent cosmetic changes of the breast following removal of the implant. Even if you have your implants replaced, implant removal may result in loss of your breast tissue. Also, implant replacement may increase your risks of future complications. For example, the risks of severe capsular contracture and reoperation double for patients with implant replacement compared to first time placement. You should consider the possibility of having your implants replaced and its consequences when making your decision to have implants.

### What are Other Potential Complications?

### Unsatisfactory Results

Unsatisfactory results such as wrinkling, asymmetry, implant displacement (shifting), incorrect size, unanticipated shape, implant palpability, scar deformity, and/or hypertrophic scarring, may occur. Some of these results may cause discomfort. Pre-existing asymmetry may not be entirely correctable by implant surgery. Revision surgery may be recommended to maintain patient satisfaction, but carries additional considerations and risks. Selecting an experienced plastic surgeon may minimize, but not necessarily prevent, unsatisfactory results.

### Pain

Pain of varying intensity and length of time may occur and persist following breast implant surgery. In addition, improper size, placement, surgical technique, or capsular contracture may result in pain. You should tell your surgeon about significant pain or if you pain persists.

In INAMED's Core Study, moderate to severe breast pain occurred in about 8% of primary and revision-augmentation patients through 4 years with most breast pain lasting about one month.

### Changes In Nipple And Breast Sensation

Feeling in the nipple and breast can increase or decrease after implant surgery. The range of changes varies from intense sensitivity to no feeling in the nipple or breast following surgery. While some of these changes can be temporary, they can also be permanent, and may affect your sexual response or your ability to nurse a baby. (See the paragraph on breast feeding).

In the study, moderate to severe nipple sensation changes were reported to occur to at least once in %5 of primary augmentation patients through 4 years. There were no reports of moderate to severe nipple sensation changes for the revision-augmentation patients through 4 years.

### Infection

Infection can occur with any surgery or implant. Most infections resulting from surgery appear within a few days to weeks after the operation. However, infection is possible at any time after surgery. In addition, breast and nipple piercing procedures may increase the possibility of infection. Infections in tissue with an implant present are harder to treat than infections in tissue without an implant. If an infection does not respond to antibiotics, the implant may have to be removed, and another implant may be placed after the infection is resolved (cleared up). As with many other surgical procedures, in rare instances, toxic shock syndrome has been noted in women after breast implant surgery, and it is a life-threatening condition. Symptoms include sudden fever, vomiting, diarrhea, fainting, dizziness, and/or sunburn-like rash. A doctor should be seen immediately for diagnosis and treatment if you have these symptoms.

### Hematoma/Seroma

Hematoma is a collection of blood within the space around the implant, and a seroma is a build-up of fluid around the implant. Having a hematoma and/or seroma following surgery may result in infection and/or capsular contracture later on. Symptoms from a hematoma or seroma may include swelling, pain, and bruising. If a hematoma or seroma occurs, it will usually be soon after surgery. However, this can also occur at any time after injury to the breast. While the body absorbs small hematomas and seromas, many will require surgery, typically involving draining and potentially the use of surgical drains for proper healing. A small scar can result from surgical draining. Implant rupture also can occur from surgical draining if there is damage to the implant during the draining procedure.

### Breast Feeding

Breast feeding difficulaties have been reported following breast surgery, including breast reduction and breast augmentation. If your surgeon uses a periareolar surgical approach (an incision around the colored portion surrounding the nipple), it may further increase the chance of breast feeding difficulties.

At this time, it is not known if small amount of silicone may pass through from the breast implant silicone shell into breast milk. Although there are no current established methods for accurately detecting silicone levels in breast milk, a study measuring silicon (one component in silicon) levels did not indicate higher levels in breast milk from women with silicone-filled gel implants when compared to women without implants.

For primary augmentation patients the study through 4 years, 9 (18%) of the 51 women who attempted to breast feed following breast implantation, experienced difficulty with breast feeding. The most common difficulty was inadequate milk production. For the 13 revision-augmentation patients who attempted to breast feed after receiving implants, 2 (15%) had difficulty breast feeding with 1 due to inadequate milk production and 1 due to pain.

### Calcium Deposits in the Tissue Around the Implant

Calcium deposits can form in the tissue capsule surrounding the implant. Symptoms may include pain and firmness. Deposits of calcium can be seen on mammograms and can be mistaken for possible cancer, resulting in additional surgery for biopsy and/or removal of the implant to distinguish calcium deposits from cancer. If additional surgery is necessary to examine and/or remove calcifications, this may cause damage to the implants. Calcium deposits also occur in women who undergo breast reduction procedures, in patents who have had hematoma formation, and even in the breasts of women who have not undergone any breast surgery. The occurrence of calcium deposits increases significantly with age.

### Extrusion

Extrusion is when the breast implant comes through your skin. This may occur, for example, when you wound has not closed or when breast tissue covering your implants weakens. Radiation therapy has been reported to increase the likelihood of extrusion. Extrusion requires additional surgery and possible removal of the implant, which may result in additional scarring and/or loss of your breast tissue.

### Necrosis

Necrosis is the death of cells or tissues. This may prevent or delay wound healing and require surgical correction, which may result in additional scarring and/or loss of your breast tissue. Implant removal may also be necessary. Factors associated with increased necrosis include infection, use of steroids, smoking, chemotherapy/radiation, and excessive heat or cold therapy.

### Delayed Wound Healing

Some patients may experience a prolonged wound healing time. Delayed wound healing may increase the risk of infection, extrusion, and necrosis. Depending on the type of surgery or the incision, wound healing times may vary. You should contact your surgeon immediately if your wound does not heal within the period of time he/she has discussed with you.

### Breast Tissue Atrophy/Chest Wall Deformity

The pressure of the breast implant may cause breast tissue thinning (with increased implant visibility and palpability) and chest wall deformity. This can occur while implants are still in place or following implant removal without replacement.

Either of these conditions may result in additional surgeries and/or unacceptable dimpling/puckering of the breast.

### Lymphadenopathy

Lymphadenopathy is a chronic enlargement of the lymph nodes. A lymph node is a round mass of tissue which makes cells as part of your immune system. The lymph nodes in the arm pit (axilla) drain the breast area of fluid. Some patients with breast implants report having enlarged lymph nodes in the armpit(s). Some reports associate lymphadenopathy with both intact and ruptured silicone breast implants. There is information in the literature that shows that armpit lymph nodes from women with silicone gel implants had abnormal tissue reactions, granulomas, and the presence of silicone. This occurred in women both with ruptured and intact implants. These reports were in women who had implants from a variety of manufactures and implant models.

Sometimes the enlarged lymph nodes are painful. If they become too large or painful, the lymph node(s) may need to be surgically removed. Painful and/or enlarged lymph nodes should be reported to your doctor.

### What are Other Reported Conditions?

There have been reports of other conditions in women with breast implants. Many of these conditions have been studied to evaluate their potential association with breast implants. Although no cause and effect relationship has been established between breast implants and conditions listed below, you should be aware of these reports. Furthermore, there is the possibility of risks, yet unknown, which in the future could be determined to be associated with breast implants.

### Connective Tissue Disease

Connective tissue disease include diseases such as lupus, scleroderma, and rheumatoid arthritis. Fibromyaglia is a disorder characterized by chronic pain in the muscles and soft tissues surrounding joints, with tenderness at specific sites in the body. It is often accompanied by fatigue. There have been a number of published epidemiological studies which have looked at whether having a breast implant is associated with having a typical or defined connective tissue disease. The study size needed to conclusively rule out the risk of a rare connective tissue disease would need to be very large. Published studies taken together show that breast implants are not significantly associated with a risk of developing a specific connective tissue disease. These studies do not distinguish between women with intact and ruptured implants. Only one study evaluated specific CTD diagnoses and symptoms in women with silent ruptured versus intact implants, but it was too small to rule out a small risk.

### Rheumatological Signs and Symptoms

Reports have also been made associating silicone breast implants with various rheumatological signs and symptoms such as fatigue, exhaustion, joint pain and swelling, muscle pain and cramping, tingling, numbness, weakness, and skin rashes. Scientific expert panels and literature reports have found no evidence of a consistent pattern of signs and symptoms in women with silicone breast implants.

Having these rheumatological signs and symptoms does not necessarily mean you have a connective tissue disease. If you notice an increase in these signs or symptoms, you should consider seeing a rheumatologist to determine whether these signs or symptoms are due to a connective tissue disorder or autoimmune disease.

### Cancer

Breast Cancer—Reports in the medical literature indicate that patients with breast implants are not a greater risk than those without breast implants for developing breast cancer. Some reports have suggested that breast implants may interfere with or delay breast cancer detection by mammography and/or biopsy; however, other reports in the published medical literature indicate that breast implants neither significantly delay breast cancer detection nor adversely affect cancer survival of women with breast implants. You should discuss this with your surgeon if you are thinking about placing a breast implant in the remaining breast to balance it with the reconstructed breast.

Brain Cancer—One recent study has reported an increase incidence of brain cancer in women with breast implants as compared to the general population. The incidence of brain cancer, however, was not significantly increased in women with breast implants when compared to women who had other plastic surgeries. Another recently published review of four large studies in women with cosmetic implants concluded that the evidence does not support as association between brain cancer and breast implants.

Respiratory/Lung Cancer—One study has reported an increased incidence of respiratory/lung cancer in women with breast implants. Other studies of women have found that women who get breast implants are more likely to be current smokers than women who get breast reduction surgery or other types of cosmetic surgery.

Cervical/Vulvar Cancer—On study has reported an increased incidence of cervical/vulvar cancer in women with breast implants. The cause of this increase is unknown, but may be due to lifestyle factors.

Other Cancers—One study has reported an increased incidence of stomach cancer and leukemia in women with breast implants compared to general population. This increase was not significant when compared to women who had other types of plastic surgeries.

### Neurological

Some women with breast implants have complained of neurological symptoms (such as difficulties with vision, sensation, muscle strength, walking, balance, thinking or remembering things) or diseases (such as multiple sclerosis), which they believed are related to their implants. A scientific expert panel report found that the evidence for a neurological disease or syndrome caused by or associated with breast implants is insufficient or flawed.

### Suicide

In several studies, a higher incidence of suicide was observed in women with breast implants. The reason for the observed increase is unknown, but it was found that women with breast implants had higher rates of hospital admission due to psychiatric causes prior to surgery, as compared with women who had breast reduction or in the general population in women.

### Effects on Children

Concerns have been raised regarding potential damaging effects on children born to mothers with implants. Two studies in humans have found that the risk of birth defects overall is not increased in children born after breast implant surgery. Although low birth weight was reported in a third study, other factors (for example, lower pre-pregnancy weight) may explain this finding. One of the authors of these human studies recommend further research on infant health.

### Gel Bleed

Small quantities of low molecular weight (LMW) siloxane compounds and platinum (in neutral state), have been found to diffuse (“bleed”) through an intact implant shell. Studies on implants implanted for a long duration have suggested that such bleed may be a contributing factor in the development of capsular contracture and lymphadenopathy. Other studies have shown evidence of silicone in scar tissue capsules surrounding the implant, in axillary lymph nodes, and in distant organs, which may be due to gel bleed. The clinical significance of the presence of silicone in these tissues is unknown. Other studies have reported that certain silicones (for example, D4 and D5) and platinum leak from intact breast implants and are present in surrounding tissue. The clinical significance to humans of the presence of silicone in the body tissues is unknown. Studies have demonstrated that the low concentration of platinum contained in breast implants is in the zero valence or most biocompatible state.

A laboratory test was conducted to analyze the siloxances and platinum (used in the manufacturing process), which may bleed out of intact implants into the body. The test method was developed to represent, as closely as possible, conditions in the body surrounding an intact implant. The results indicate that both LMW siloxanes and platinum bled in the serum in very low but measurable quantities. A total of approximately 45 micrograms (45 millionths of a gram) of these LMW siloxanes bled from the implant during the 90 test. On a “whole device” basis, 99.99993% of the siloxanes were retained. A total of approximately 1.1 micrograms (1.1 millonths of a gram) of platinum bled from the implant during the 90 day test. On a “whole device” basis, 99.7% of platinum was retained.

The following section is also distributed in the binder.

### Device Implant Tracking Instructions

INAMED silicone-filled breast implants are subject to Device Tracking per federal regulation. Because your implant is a tracked device, your healthcare provided is required to report certain information to NAMED following surgery, including implant specific information like serial number and catalog number as well as the date of surgery, implanting surgeon name and contact information and patient specific information.

The Device Tracking form is a 2 part document and the top portion has been forwarded to INAMED by your surgeon. Upon receipt by INAMED of the healthcare provider portion of the Device Tracking System form, you are entered in the Device Tracking database. The patient portion of the form has been supplied to you to complete enrollment in INAMED's Device Tacking database. Your Device Tracking form should have stickers with information about your implant(s). If the stickers are not already attached, use your Patient/Device ID card supplied following surgery to enter the serial number (SN) and catalog number (REF) of your implant(s). INAMED strongly encourages you to participate in Device Tacking. As information regarding the long-term safety of silicone breast implants becomes available, your participation will allow us to provide this information to you. If you do NOT wish to participate in Device Tracking, check the indicated box and INAMED will remove your personal information from the database.

Complete the section titled REQURIED INFORMATION. This section does NOT affect your decision to participate or not participate in Device Tracking. Your response here will help INAMED assess the effectiveness of this patient planner.

Place your completed Device Tracking form in the envelope provided and return to INAMED Aesthetics.

### Device Tracking

Silicone-filled breast implants are subject to Device Tracking by Federal regulation. This means that your physician will be required to report to INAMED the serial number of the device(s) you receive, the date of surgery, and information relating to the physician's practice. This information will be recorded on the Device Tracking Form supplied by INAMED with each silicone-filled breast implant. Your surgeon will return the top portion of the form to INAMED following surgery. The bottom portion of the form will be provided to you following surgery. You have the right to refuse to release your personal information for the purpose of device tracking. If you choose NOT to participate in Device Tracking please check the appropriate box on the form supplied. If you elect to participate, complete the section titled “Patient Information”. In order to assess the effectiveness of the information you were provided prior to your surgery you will also fill out the “Required Information” section on the form. This section asks you to verify that you received and had adequate time to review this patient labeling information.

INAMED strongly recommends that all patients receiving silicone-filled breast implants be registered in the Device Tracking database. This will help ensure that INAMED has a record of each patient's contact information so that they may continue to be updated with the latest available information regarding their silicone-filled breast implants.

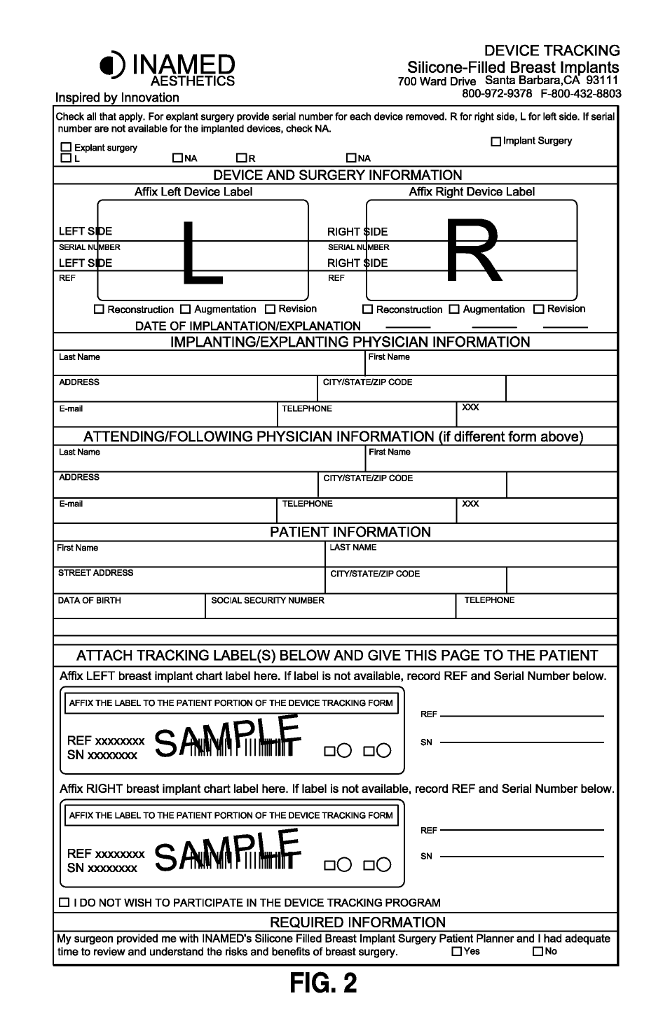

You are encouraged to complete the Device Tacking Form (See FIG. 2) you received following surgery and return it to INAMED Corporation in the postage paid business replay envelope provided.

### Additional Information

### Device Identification Card

You will also be given a device identification card with the style and serial number of your breast implant(s). This card is for your permanent record and should be kept in a safe place. In the event you have a concern or problem with your implant you can use this card to describe the implant to your health care provider or to INAMED.

### ConfidencePlus™ Limited Warranties

The ConfidencePlus™ Limited Warranties provide lifetime replacement and limited financial reimbursement in the event of shell leakage or breakage resulting in implant rupture, subject to certain conditions as fully discussed in the ConfidencePlust™ literature. INAMED offers two levels of coverage under its warranty program. Our standard ConfidencePlus™ Limited Warranty programs applies automatically to every INAMED breast implant recipient subject to the conditions discussed in the ConfidencePlus™ literature. The optional ConfidencePlus™ Platinum Limited Warranty program is available for a low enrollment fee and increases the financial benefit in the event of implant rupture, subject to the conditions discussed in the ConfidencePlus™ literature.

The following sections provides for recording of surgery data, location and contact person.

### Information for the Mammography Center

Please update my patient file to reflect the presence of INAMED silicone-filled breast implants. Since examination of the augmented breasts is more time consuming please allow additional time when scheduling my next mammogram and alert the physician and technologist performing the exam about the presence of my implants. When performing my mammography please use diagnostic rather than screening techniques and take additional views (called Eklund views or implant displacement (ID) views) to ensure adequate imaging of the breast tissue.

Silicone-filled breast implants have been approved by the FDA for use in augmentation, reconstruction, and revision surgery. As part of a woman's health care nextwork, it's important that you are aware of the latest information on the safety of silicone breast implants. For additional information please consider the following resources:

### Information for you Primary Care Physician

Your patient has been implanted with NAMED silicone-filled breast implants. It is important that you include this information in her chart because while silicone-filled breast implants have been proven safe in thousands of patients worldwide, they can present additional challenges for attending physicians. So to ensure your patient receives the care she needs, when appropriate please alert other physicians about the presence of her implants.

You may be aware that the FDA has approved silicone-filled breast implants for use in augmentation, reconstruction and revision surgery. This approval is the result of decades of research exploring and confirming the safety of silicone implants. As part of a woman's health care network, we invite you to make yourself aware of the latest information on the use of silicone breast implants in augmentation, reconstruction and revision surgery and why, for many women, it is the best surgical option.

The planner 1 includes all of the hereinabove presented sections in a single bound volume to improve its implementation and increase the likelihood of consistent discussion with the patient.

Although there has been hereinabove described a specific diary/planner for breast surgery preparation and method in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. A method for a patient to assess risks of breast augmentation surgery wherein the patient is considering breast augmentation surgery, the method comprising the following steps carried out by the patient:
(a) reading a list of surgical risks of breast implant;
(b) reading a self-assessment form including a series of true and false statements;
(c) marking on the form whether each statement is true or false to assess the patient's understanding of said listed surgical risks; and
(d) one to two weeks after step (c), meeting with a surgeon and marking a series of statements on a patient understanding form containing at least some of the listed surgical risks, the patient understanding form including initial blocks for both patient and surgeon.

2. The method of claim 1 further comprising (e) after completing step (d), signing a surgical consent form with signature blocks for both patient and surgeon.

3. The method of claim 1 wherein the list of surgical risks read by the patient includes risks relating to capsular contracture, reoperations, implant removal, unsatisfactory results, changes in nipple and breast sensation, infection, hemetoma/serona, breast feeding, calcium deposits, extrusion, delayed wound healing, breast tissue atrophy, lymphadenopalty, connective tissue disease, rheumatological signs, cancer, neurological symptoms, effects on children, and gel bleed.

4. The method of claim 1 wherein the series of statements of the self-assessment form includes statements relating to suspected rupture, reoperations; implant removal, mammography, breastfeeding, self examination, autoimmune diseases, weakened immune system, gel migration, and capsular contracture.

5. The method of claim 1 wherein the series of statements of the self-assessment form includes the statements:
1) “In the event of a suspected rupture, you should see your physician and in consultation with your physician, you should consider removal of the implant(s).”
2) “Additional surgery to your breast and/or implant will be likely over the course of your life.”
3) “Your implants are not considered lifetime devices and you will likely undergo implant removal, with or without replacement, during your life.”
4) “You should inform your mammographers about the presence of your implants.”
5) “Your breast implants may interfere with your ability to successfully breastfeed.”
6) “You should perform breast self-examinations monthly and should make sure you know how to distinguish the implant from your breast tissue.”
7) “Silicone breast implants have not been clinically tested in women with autoimmune diseases like lupus or scleroderma.”
8) “If you have serious health problems or conditions such as weakened immune system or compromised blood supply to the breast you should discuss with your surgeon whether breast augmentation surgery is appropriate for you.”
9) “To detect possible ‘silent rupture’ an MRI is recommended 3 years following initial surgery and every two years thereafter.”
10) “Although rare, there have been reports in the scientific literature providing evidence that the silicone gel fill may move beyond the fibrous capsule and into the breast tissue or away form the breast (gel migration), particularly if the scar capsule is ruptured, causing local complications such as pain and neuropathy.”
11) “Capsular contracture or hardening of the tissue surrounding the breast implant may result in the need for additional surgery.”
